SEQUENCE LISTING (4 sequences)

SEQ ID NO: 1 - DNA, 1695 bp, Homo sapiens
atgtggttctgcgcgtgtgcggacggctgtctgttaactccgcggtcagttcccggactggtggctggtctgcagggttgacctgcgcaatgcagaggctgcaggtagtgctgggccacctgaggggtccggccgattccggctggatgccgcaggccgcgccttgcctgagcggtgccccgcaggcctcggccgcggacgtggtggtggtgcacgggcggcgcacggccatctgccgggcgggccgcggcggcttcaaggacaccacccccgacgagcttctctcggcagtcatgaccgcggttctcaaggacgtgaatctgaggccggaacagctgggggacatctgtgtcggaaatgtgctgcagcctggggccggggcaatcatggcccgaatcgcccagtttctgagtgacatcccggagactgtgcctttgtccactgtcaatagacagtgttcgtcggggctacaggcagtggccagcatagcaggtggcatcagaaatgggtcttatgacattggcatggcctgtggggtggagtccatgtccctggctgacagagggaaccctggaaatattacttcgcgcttgatggagaaggagaaggccagagattgcctgattcctatggggataacctctgagaatgtggctgagcggtttggcatttcacgggagaagcaggatacctttgccctggcttcccagcagaaggcagcaagagcccagagcaagggctgtttccaagctgagattgtgcctgtgaccaccacggtccatgatgacaagggcaccaagaggagcatcactgtgacccaggatgagggtatccgccccagcaccaccatggagggcctggccaaactgaagcctgccttcaagaaagatggttctaccacagctggaaactctagccaggtgagtgatggggcagctgccatcctgctggcccggaggtccaaggcagaagagttgggccttcccatccttggggtcctgaggtcttatgcagtggttggggtcccacctgacatcatgggcattggacctgcctatgccatcccagtagctttgcaaaaagcagggctgacagtgagtgacgtggacatcttcgagatcaatgaggcctttgcaagccaggctgcctactgtgtggagaagctacgactcccccctgagaaggtgaaccccctggggggtgcagtggccttagggcacccactgggctgcactggggcacgacaggtcatcacgctgctcaatgagctgaagcgccgtgggaagagggcatacggagtggtgtccatgtgcatcgggactggaatgggagccgctgccgtctttgaataccctgggaactgagtgaggtcccaggctggaggcgctacgcagacagtcctgctgctctagcagcaaggcagtaacaccacaaaagcaaaaccacatgggaaaactcagcactggtggtggtggcagtggacagatcaaggcacttcaactcatttggaaaatgtgaacactgatgacatggtataggagtgggtggggtgttgagccacccatcagaccctctttagctgtgcaagataaaagcagcctgggtcacccaggccacaaggccatggttaattcttaaggcaaggcaaatccatggatgagaagtgcaatgggcatagtaaaagtgcatgaattt

SEQ ID NO: 2 - protein, 424 aa, Homo sapiens
MQRLQVVLGHLRGPADSGWMPQAAPCLSGAPQASAADVVVVHGRRTAICRAGRGGFKDTTPDELLSAVMTAVLKDVNLRPEQLGDICVGNVLQPGAGAIMARIAQFLSDIPETVPLSTVNRQCSSGLQAVASIAGGIRNGSYDIGMACGVESMSLADRGNPGNITSRLMEKEKARDCLIPMGITSENVAERFGISREKQDTFALASQQKAARAQSKGCFQAEIVPVTTTVHDDKGTKRSITVTQDEGIRPSTTMEGLAKLKPAFKKDGSTTAGNSSQVSDGAAAILLARRSKAEELGLPILGVLRSYAVVGVPPDIMGIGPAYAIPVALQKAGLTVSDVDIFEINEAFASQAAYCVEKLRLPPEKVNPLGGAVALGHPLGCTGARQVITLLNELKRRGKRAYGVVSMCIGTGMGAAAVFEYPGN

SEQ ID NO: 3 - DNA, 1952 bp, Homo sapiens
gtgtttagggtgttggcggagacaaaggggaagagtcatcgcctgtcggggctaggatatgatgggtgagaggtgtcaaaccaaattctctcggtttggaaacggagaaaatctaaaaatgaggatgtgaggaaagagtccgctctcaaggcgcgttgtggtctatccgagccccgtcccctgggctccctcgggctggggtgaggcgggcagcgctacgcgtggggtaggaccatcttacgctgggaccccgccaaggagccccaggaagtaggtgaaagggcaggggcgtggctctcggggcgccacccacgctcttgaaatctgggtgattgcgagcggccgctcagcgtcccccacaccacagacccgcgccgccgacgacccagcagccgccatggctctgctccgaggtgtgtttgtagttgctgctaagcgaacgccctttggagcttacggaggccttctgaaagacttcactgctactgacttgtctgaatttgctgccaaggctgccttgtctgctggcaaagtctcacctgaaacagttgacagtgtgattatgggcaatgtcctgcagagttcttcagatgctatatatttggcaaggcatgttggtttgcgtgtgggaatcccaaaggagaccccagctctcacgattaataggctctgtggttctggttttcagtccattgtgaatggatgtcaggaaatttgtgttaaagaagctgaagttgttttatgtggaggaaccgaaagcatgagccaagctccctactgtgtcagaaatgtgcgttttggaaccaagcttggatcagatatcaagctggaagattctttatgggtatcattaacagatcagcatgtccagctccccatggcaatgactgcagagaatcttgctgtaaaacacaaaataagcagagaagaatgtgacaaatatgccctgcagtcacagcagagatggaaagctgctaatgatgctggctactttaatgatgaaatggcaccaattgaagtgaagacaaagaaaggaaaacagacaatgcaggtagacgagcatgctcggccccaaaccaccctggaacagttacagaaacttcctccagtattcaagaaagatggaactgttactgcagggaatgcatcgggtgtagctgatggtgctggagctgttatcatagctagtgaagatgctgttaagaaacataacttcacaccactggcaagaattgtgggctactttgtatctggatgtgatccctctatcatgggtattggtcctgtccctgctatcagtggggcactgaagaaagcaggactgagtcttaaggacatggatttggtagaggtgaatgaagcttttgctccccagtacttggctgttgagaggagtttggatcttgacataagtaaaaccaatgtgaatggaggagccattgctttgggtcacccactgggaggatctggatcaagaattactgcacacctggttcacgaattaaggcgtcgaggtggaaaatatgccgttggatcagcttgcattggaggtggccaaggtattgctgtcatcattcagagcacagcctgaagagaccagtgagctcactgtgacccatccttactctacttggccaggccacagtaaaacaagtgaccttcagagcagctgccacaactggccatgccctgccattgaaacagtgattaagtttgatcaagccatggtgacacaaaaatgcattgatcatgaataggagcccatgctagaagtacattctctcagatttgaaccagtgaaatatgatgtatttctgagctaaaactcaactatagaagacattaaaagaaatcgtattcttgccaagtaaccaccacttctgccttagataatatgattataaggaaatcaaataaatgttgccttaacttcaaaaaaaaaaaaaaaaaaa

SEQ ID NO: 4 - protein, 397 aa, Homo sapiens
MALLRGVFVVAAKRTPFGAYGGLLKDFTATDLSEFAAKAALSAGKVSPETVDSVIMGNVLQSSSDAIYLARHVGLRVGIPKETPALTINRLCGSGFQSIVNGCQEICVKEAEVVLCGGTESMSQAPYCVRNVRFGTKLGSDIKLEDSLWVSLTDQHVQLPMAMTAENLAVKHKISREECDKYALQSQQRWKAANDAGYFNDEMAPIEVKTKKGKQTMQVDEHARPQTTLEQLQKLPPVFKKDGTVTAGNASGVADGAGAVIIASEDAVKKHNFTPLARIVGYFVSGCDPSIMGIGPVPAISGALKKAGLSLKDMDLVEVNEAFAPQYLAVERSLDLDISKTNVNGGAIALGHPLGGSGSRITAHLVHELRRRGGKYAVGSACIGGGQGIAVIIQSTA

(19) United States
(12) Patent Application Publication
(10) Pub. No.: US 2011/0150773 A1
(43) Pub. Date: Jun. 23, 2011

(54) MODULATORS OF ACETYL-COENZYME A ACYLTRANSFERASE 1 OR 2 IN THE TREATMENT OF ACNE, OF SEBORRHOEIC DERMATITIS OR OF HYPERSEBORRHOEA

(75) Inventors: Jérôme Aubert, Grasse (FR); Michel Rivier, Nice (FR); Johannes Voegel, Chateauneuf/Grasse (FR)

(73) Assignee: GALDERMA RESEARCH & DEVELOPMENT, Biot (FR)

(21) Appl. No.: 12/991,060

(22) PCT Filed: May 7, 2009
(86) PCT No.: PCT/EP09/55556

§ 371 Date: Mar. 7, 2011

Related U.S. Application Data
(60) Provisional application No. 61/071,609, filed on May 7, 2008.

(30) Foreign Application Priority Data
Nov. 13, 2008 (FR) 0857709

(51) Int. Cl. A61K 49/00; G01N 33/53; C12Q 1/68; A61K 39/395; C07H 21/00; C12N 9/10; A61P 17/08; A61P 17/10

(52) U.S. Cl. 424/9.2; 435/7.8; 435/6.18; 424/146.1; 435/7.92; 536/23.2; 435/193

(57) ABSTRACT

An in vitro method for screening for candidate compounds for the preventive or curative treatment of acne, of seborrhoeic dermatitis or of skin disorders associated with hyperseborrhoea, includes determining the ability of a compound to modulate the expression or the activity of acetyl-coenzyme A acyltransferase 1 (ACAA1) or of acetyl-coenzyme A acyltransferase 2 (ACAA2), and also utilizes modulators of the expression or of the activity of either of these enzymes, for the treatment of acne, of seborrhoeic dermatitis or of skin disorders associated with hyperseborrhoea; methods for the in vitro diagnosis of or in vitro prognosis for these pathologies are also featured.

The invention relates to the identification and the use of compounds which modulate acetyl-coenzyme A acyltransferase 1 (ACAA1) or acetyl-coenzyme A acyltransferase 2 (ACAA2) for treating acne, seborrhoeic dermatitis, and also skin disorders associated with hyperseborrhoea. It also relates to methods for the in vitro diagnosis of or in vitro prognosis for these pathologies.

Hyperseborrhoeic greasy skin is characterized by exaggerated secretion and excretion of sebum. Conventionally, a sebum level greater than 200 μg/cm² measured on the forehead is considered to be characteristic of greasy skin. Greasy skin is often associated with a desquamation deficiency, a glistening complexion and a thick skin grain. In addition to these aesthetic disorders, excess sebum can serve as a support for the anarchical development of saprophytic bacterial flora (P. acnes in particular), and cause the appearance of comedones and/or acneic lesions.

This stimulation of sebaceous gland production is induced by androgens.

Acne is, in fact, a chronic disease of the pilosebaceous follicle under hormonal control. Hormone therapy against acne is one treatment possibility for women, the objective being to prevent the effects of androgens on the sebaceous gland. In this context, oestrogens, anti-androgens or agents which reduce the production of androgens by the ovaries or the adrenal gland are generally used. The anti-androgens used for the treatment of acne include, in particular, spironolactone, cyproterone acetate and flutamide. However, these agents have potentially severe side effects. Thus, any pregnancy must be absolutely prevented, in particular because of a risk of feminization for the male foetus. These agents are prohibited in male patients.

Seborrhoeic dermatitis is a common inflammatory skin dermatosis which presents in the form of red plaques covered with greasy, yellowish squames, which are more or less pruriginous, and are predominant in the seborrhoeic areas.

A need therefore exists, for these diseases, to identify mediators downstream of the action of the steroid hormones, and to modulate them, in order to obtain a similar therapeutic profile, but with reduced side effects.

The Applicant has now discovered that the genes encoding acetyl-coenzyme A acyltransferase 1 (ACAA1) or acetyl-coenzyme A acyltransferase 2 (ACAA2) are expressed preferentially in human sebaceous glands in comparison with the epidermis, and that the expression thereof is regulated in vitro by a cocktail which promotes the differentiation of sebocyte precursors, containing an androgen (R1881, also known as methyltrienolone, at 1 nM) and a PPARγ ligand (Rosiglitazone, which is 6-(2-methoxyethoxy-methoxy)naphthalene-2-carboxylic acid [4′-(2,4-dioxothiazolidin-5-ylmethyl)biphenyl-3-ylmethyl]methyl-amide, 100 nM), in a primary culture of human sebocytes.

It consequently proposes targeting the ACAA1 or ACAA2 genes or the expression product thereof, for preventing and/or improving acne, seborrhoeic dermatitis or skin disorders associated with hyperseborrhoea, in particular the greasy skin appearance.

The Applicant also demonstrates that these targets are present in an animal pharmacology model (Fuzzy rat), this model being relevant for the acne pathology and hyperseborrhoea (Ye et al., 1997, Skin Pharmacol, 10(5-6):288-97).

It is, moreover, known that treatment with a PPAR agonist induces a large decrease in the size of the sebaceous glands, and a reduction in androgen-induced hyperseborrhoea (WO2007/093747).

Since the targets proposed are downstream of the PPAR receptor, it is said targets which are responsible for the effects observed on the sebaceous glands and on sebum excretion.

Thus, the genes identified, which act downstream of the PPAR receptor, can be used to identify the compounds which are the most active as PPAR modulators, to classify them and to select them. On this basis, it is also proposed to use the ACAA1 or ACAA2 genes, or the ACAA1 or ACAA2 protein thereof, as markers for screening for candidate PPAR modulators for the treatment of acne, seborrhoeic dermatitis or skin disorders associated with hyperseborrhoea. More specifically, the ability of a PPAR modulator to modulate the expression or the activity of ACAA1 or ACAA2 or the expression of the gene thereof or the activity of at least one of the promoters thereof, can be determined.

The term “acne” is intended to mean all the forms of acne, i.e. in particular acne vulgaris, comedonal acne, polymorphous acne, nodulocystic acne, acne conglobata, or else secondary acne such as solar acne, acne medicamentosa or occupational acne. The Applicant also proposes methods of in vitro, in vivo and clinical diagnosis or prognosis based on the detection of the level of expression or of activity of ACAA1 or of ACAA2.

### ACAA1

The ACAA1 enzyme denotes acetyl-coenzyme A acyltransferase 1, also known as mitochondrial oxoacyl-CoA thiolase 1,3-ketoacyl-CoA thiolase, peroxisomal precursor (EC 2.3.1.16) (beta-ketothiolase) (acetyl-CoA acyltransferase) (peroxisomal 3-oxoacyl-CoA thiolase).

The acetyl-coenzyme A acyltransferase 1 gene was identified by Bout et al. (1991, Biochim Biophys Acta, 1090(1):43-51) and encodes an enzyme which cleaves 3-ketoacyl-CoA to give acetyl-CoA and acyl-CoA during the fatty acid beta-oxidation cycle which takes place in the peroxisome. The gene encoding acetyl-coenzyme A acyltransferase 1 is, in the context of the present application, referred to as ACAA1 gene. In the peroxisome, at least two thiolase enzymes catalyse the final stage of the beta-oxidation: ACAA1 and SCP-2 (Antonenkov, 1997, 272(41); 26023-26031). Various studies have demonstrated symptoms associated with a potential ACAA1 deficiency (Schram et al., PNAS, 1987, 84(8):2494-6, Goldfinger et al., 1986, J Pediatr., 108(1):25-32). Finally, a more recent study carried out in 2002 in a patient exhibiting an accumulation of very-long-chain fatty acids, as described in the previous studies, did not confirm an ACAA1 deficiency (Ferdinandusse et al., 2002, Am. J. Hum Genet. 70:1589-1593).

### ACAA2

The ACAA2 enzyme denotes acetyl-coenzyme A acyltransferase 2, also known as 3-ketoacyl-CoA thiolase, mitochondrial (EC 2.3.1.16) (beta-ketothiolase) (acetyl-CoA acyltransferase) (mitochondrial 3-oxoacyl-CoA thiolase).

Acetyl-coenzyme A acyltransferase 2 cleaves 3-ketoacyl-CoA to give acetyl-CoA and acyl-CoA during the fatty acid beta-oxidation cycle which takes place in the mitochondrion. The gene encoding acetyl-coenzyme A acyltransferase 2 is, in the context of the present application, referred to as ACAA2 gene. The ACAA2 gene has been proposed as a target in the treatment of cardiac insufficiency (Lopaschuk, et al. 2003, Circ Res. August 8; 93(3):e33-7) in particular in the case of diabetes. During cardiac insufficiency, the inhibition of ACAA2 with trimetazidine, for example, would make it possible to decrease an excessive level of fatty acid oxidation in the myocardium and would thus promote the contractile function of the heart (Onay-Besikci A, et al., 2007, Can J Physiol. Pharmacol.; 85(5):527-35). In addition, it has been shown that activated PPARα receptors can increase the expression of beta-oxidation enzymes, including ACAA2 (Aoyama et al., 1998, The Journal of Biological Chemistry, Vol. 276, pp 5678-84).

In the context of the invention, the terms “ACAA1 gene” or “ACAA2 gene” and “ACAA1 nucleic acid” or “ACAA2 nucleic acid” signify the genes or the nucleic acid sequences which encode acetyl-coenzyme A acyltransferase 1 or acetyl-coenzyme A acyltransferase 2. While the targets aimed for are preferably the human genes or the expression product thereof, the invention may also call upon cells expressing a heterologous acetyl-coenzyme A acyltransferase 1 or heterologous acetyl-coenzyme A acyltransferase 2, by genomic integration or transient expression of an exogenous nucleic acid encoding the enzyme.

A human cDNA sequence of ACAA1 is reproduced in the annexe (SEQ ID No. 1). It is the sequence NM_{—}001607 (Genbank), the open reading frame of which contains 1695 base pairs and encodes an amino acid sequence of 424 residues.

A human cDNA sequence of ACAA2 is reproduced in the annexe (SEQ ID No. 3). It is the sequence NM_{—}006111 (Genbank), the open reading frame of which contains 1191 base pairs and encodes an amino acid sequence of 397 residues.

### Diagnostic Applications

A subject of the invention concerns an in vitro method for diagnosing or monitoring the development of acneic lesions, seborrhoeic dermatitis or a skin disorder associated with hyperseborrhoea in an individual, comprising the comparison of the expression or of the activity of the acetyl-coenzyme A acyltransferase 1 (ACAA1) or acetyl-coenzyme A acyltransferase 2 (ACAA2) proteins, of the expression of the gene thereof or of the activity of at least one promoter thereof, in a biological sample from an individual, with respect to a biological sample from a control individual.

The protein expression can be determined by assaying the ACAA1 or ACAA2 protein according to one of the methods such as Western blotting, immunohistochemistry, mass spectrometry analysis (Maldi-TOF and LC/MS analysis), radioimmunoassay (RIA) or ELISA or any other method known to those skilled in the art. Another method, in particular for measuring the expression of the ACAA1 or ACAA2 genes, is to measure the amount of corresponding mRNA. Assaying the activity of the ACAA1 or ACAA2 proteins can also be envisaged.

In the context of a diagnosis, the “control” individual is a “healthy” individual.

In the context of monitoring the development of acneic lesions, of seborrhoeic dermatitis or of a skin disorder associated with hyperseborrhoea, the “control individual” refers to the same individual at a different time, which preferably corresponds to the beginning of the treatment (T0). This measurement of the difference in expression or in activity of ACAA1 or ACAA2, or in expression of the gene thereof or in activity of at least one promoter thereof, makes it possible in particular to monitor the effectiveness of a treatment, in particular a treatment with an ACAA1 or ACAA2 modulator, as envisaged above, or another treatment against acne, seborrhoeic dermatitis or a skin disorder associated with hyperseborrhoea. Such monitoring can reassure the patient with regard to whether continuing the treatment is well-founded or necessary.

Another aspect of the present invention concerns an in vitro method for determining an individual's susceptibility to developing acneic lesions, seborrhoeic dermatitis or a skin disorder associated with hyperseborrhoea, comprising the comparison of the expression or of the activity of the acetyl-coenzyme A acyltransferase 1 or acetyl-coenzyme A acyltransferase (ACAA1 or ACAA2) proteins, of the expression of the gene thereof or of the activity of at least one of the promoters thereof, in a biological sample from an individual, with respect to a biological sample from a control individual.

Here again, the expression of the ACAA1 or ACAA2 proteins can be determined by assaying this protein by immunoassay, for example by ELISA assay, or by any other method mentioned above. Another method, in particular for measuring the expression of the ACAA1 or ACAA2 genes, is to measure the amount of corresponding mRNA by any method as described above. Assaying of the ACAA1 or ACAA2 activity can also be envisaged.

The individual tested is in this case an asymptomatic individual exhibiting no skin condition associated with hyperseborrhoea, seborrhoeic dermatitis or acne. The “control” individual in this method signifies a “healthy” reference population or individual. The detection of this susceptibility makes it possible to set up a preventive treatment and/or increased monitoring of the signs associated with acne, seborrhoeic dermatitis or a skin disorder associated with hyperseborrhoea.

In these in vitro diagnostic or prognostic methods, the biological sample tested may be any sample of biological fluid or a sample of a biopsy. Preferably, the sample may be a preparation of skin cells, obtained for example by desquamation or biopsy. It may also be sebum.

### Screening Methods

A subject of the invention is an in vitro or in vivo method for screening for candidate compounds for the preventive and/or curative treatment of acne, of seborrhoeic dermatitis or of any skin disorder associated with hyperseborrhoea, comprising the determination of the ability of a compound to modulate the expression or the activity of acetyl-coenzyme A acyltransferase 1 or acetyl-coenzyme A acyltransferase 2 or the expression of the gene thereof or the activity of at least one of the promoters thereof, said modulation indicating the usefulness of the compound for the preventive or curative treatment of acne, seborrhoeic dermatitis or any skin disorder associated with hyperseborrhoea. The method therefore makes it possible to select the compounds capable of modulating the expression or the activity of ACAA1 or of ACAA2, or the expression of the gene thereof, or the activity of at least one of the promoters thereof.

More particularly, the subject of the invention is an in vitro method for screening for candidate compounds for the preventive and/or curative treatment of acne, of seborrhoeic dermatitis or of skin disorders associated with hyperseborrhoea, comprising the following steps:
a. preparing at least two biological samples or reaction mixtures;
b. bringing one of the samples or reaction mixtures into contact with one or more of the test compounds;
c. measuring the expression or the activity of the acetyl-coenzyme A acyltransferase 1 or 2 proteins, the expression of the gene thereof or the activity of at least one of the promoters thereof, in the biological samples or reaction mixtures;
d. selecting the compounds for which a modulation of the expression or of the activity of the acetyl-coenzyme A acyltransferase 1 or acetyl-coenzyme A acyltransferase 2 proteins, of the expression of the gene thereof or of the activity of at least one of the promoters thereof, is measured in the sample or the mixture treated in b), compared with the untreated sample or with the untreated mixture.

An in vivo screening method can be carried out in any laboratory animal, for example, a rodent. According to one preferred embodiment, the screening method comprises administering the test compound to the animal preferably by topical application, then optionally sacrificing the animal by euthanasia, and taking a sample of an epidermal split, before evaluating the expression of the gene in the epidermal split, by any method described herein.

The term “modulation” is intended to mean any effect on the expression or the activity of the enzyme, the expression of the gene or the activity of at least one of the promoters thereof, i.e. optionally a stimulation, but preferably a partial or complete inhibition. Thus, the compounds tested in step d) above preferably inhibit the expression or the activity of the acetyl-coenzyme A acyltransferase 1 or acetyl-coenzyme A acyltransferase 2 proteins, the expression of the gene thereof or the activity of at least one of the promoters thereof. The difference in expression obtained with the compound tested, compared with a control carried out in the absence of the compound, is significant starting from 25% or more.

Throughout the present text, unless otherwise specified, the term “expression of a gene” is intended to mean the amount of mRNA expressed;

the term “expression of a protein” is intended to mean the amount of this protein;

the term “activity of a protein” is intended to mean the biological activity thereof;

the term “activity of a promoter” is intended to mean the ability of this promoter to initiate the transcription of the DNA sequence encoded downstream of this promoter (and therefore indirectly the synthesis of the corresponding protein).

The compounds tested may be of any type. They may be of natural origin or may have been produced by chemical synthesis. This may involve a library of structurally defined chemical compounds, uncharacterized compounds or substances, or a mixture of compounds.

In particular, the invention is directed towards the use of ACAA1 or ACAA2 genes or of the protein thereof, as a marker for candidate PPAR or AR (androgen receptor) modulators for treating acne, seborrhoeic dermatitis or a skin disorder associated with hyperseborrhoea. More specifically, the ability of a PPAR or AR modulator to modulate the expression or the activity of ACAA1 or of ACAA2 or the expression of the gene thereof or the activity of at least one of the promoters thereof is determined. Preferably, the modulator is a PPARγ modulator.

The PPAR modulator is a PPAR agonist or antagonist, preferably an agonist.

The AR modulator is an AR agonist or antagonist, preferably an agonist.

Various techniques can be used to test these compounds and to identify the compounds of therapeutic interest which modulate the expression or the activity of acetyl-coenzyme A acyltransferase 1 or acetyl-coenzyme A acyltransferase 2.

According to a first embodiment, the biological samples are cells transfected with a reporter gene functionally linked to all or part of the promoter of the gene encoding acetyl-coenzyme A acyltransferase 1 or acetyl-coenzyme A acyltransferase 2, and step c) described above comprises measuring the expression of said reporter gene.

The reporter gene may in particular encode an enzyme which, in the presence of a given substrate, results in the formation of coloured products, such as CAT (chloramphenicol acetyltransferase), GAL (beta-galactosidase) or GUS (beta-glucuronidase). It may also be the luceriferase gene or the GFP (green fluorescent protein) gene. The assaying of the protein encoded by the reporter gene, or of the activity thereof, is carried out conventionally by colorimetric, fluorometric or chemiluminescence techniques, inter alia.

According to a second embodiment, the biological samples are cells expressing the gene encoding acetyl-coenzyme A acyltransferase 1 or acetyl-coenzyme A acyltransferase 2, and step c) described above comprises measuring the expression of said gene.

The cell used herein may be of any type. It may be a cell expressing the ACAA1 or ACAA2 gene endogenously, for instance a liver cell, an ovarian cell, or better still a sebocyte. Organs of human or animal origin may also be used, for instance the preputial gland, the clitoral gland, or else the sebaceous gland of the skin.

It may also be a cell transformed with a heterologous nucleic acid encoding preferably human, or mammalian, acetyl-coenzyme A acyltransferase 1 or acetyl-coenzyme A acyltransferase 2.

A large variety of host-cell systems may be used, such as, for example, Cos-7, CHO, BHK, 3T3 or HEK293 cells. The nucleic acid may be transfected stably or transiently, by any method known to those skilled in the art, for example by calcium phosphate, DEAE-dextran, liposome, virus, electroporation or microinjection.

In these methods, the expression of the ACAA1 or ACAA2 genes or of the reporter gene can be determined by evaluating the level of transcription of said gene, or the level of translation thereof.

The expression “level of transcription of a gene” is intended to mean the amount of corresponding mRNA produced. The expression “level of translation of a gene” is intended to mean the amount of protein produced. Those skilled in the art are familiar with the techniques for quantitatively or semi-quantitatively detecting the mRNA of a gene of interest. Techniques based on hybridization of the mRNA with specific nucleotide probes are the most common (Northern blotting, RT-PCR (reverse transcriptase polymerase chain reaction), quantitative RT-PCR (qRT-PCR), RNase protection). It may be advantageous to use detection labels, such as fluorescent, radioactive or enzymatic agents or other ligands (for example, avidin/biotin).

In particular, the expression of the genes can be measured by real-time PCR or by RNase protection. The term “RNase protection” is intended to mean the detection of a known mRNA among the poly(A)-RNAs of a tissue, which can be carried out using specific hybridization with a labelled probe. The probe is a labelled (radioactive) RNA complementary to the messenger to be sought. It can be constructed from a known mRNA, the cDNA of which, after RT-PCR, has been cloned into a phage. Poly(A)-RNA from the tissue in which the sequence is to be sought is incubated with this probe under slow hybridization conditions in a liquid medium. RNA:RNA hybrids form between the mRNA sought and the antisense probe. The hybridized medium is then incubated with a mixture of ribonucleases specific for single-stranded RNA, such that only the hybrids formed with the probe can withstand this digestion. The digestion product is then deproteinated and repurified, before being analysed by electrophoresis. The labelled hybrid RNAs are detected by autoradiography.

The level of translation of the gene is evaluated, for example, by immunological assaying of the product of said gene. The antibodies used for this purpose may be of polyclonal or monoclonal type. The production thereof involves conventional techniques. An anti-ACAA1 or anti-ACAA2 polyclonal antibody can, inter alia, be obtained by immunization of an animal, such as a rabbit or a mouse, with the whole enzyme. The antiserum is taken and then depleted according to methods known per se to those skilled in the art. A monoclonal antibody can, inter alia, be obtained by the conventional method of Köhler and Milstein (Nature (London), 256: 495-497 (1975)). Other methods for preparing monoclonal antibodies are also known. Monoclonal antibodies can, for example, be produced by expression of a nucleic acid cloned from a hybridoma. Antibodies can also be produced by the phage display technique, by introducing antibody cDNAs into vectors, which are typically filamentous phages which display V-gene libraries at the surface of the phage (for example, fUSES for E. coli).

The immunological assaying can be carried out in solid phase or in homogeneous phase; in one step or in two steps; in a sandwich method or in a competition method, by way of nonlimiting examples. According to one preferred embodiment, the capture antibody is immobilized on a solid phase. By way of nonlimiting examples of a solid phase, use may be made of microplates, in particular polystyrene microplates, or solid particles or beads, or paramagnetic beads.

ELISA assays, radioimmunoassays or any other detection technique can be used to reveal the presence of the antigen/antibody complexes formed.

The characterization of the antigen/antibody complexes, and more generally of the isolated or purified, but also recombinant, proteins (obtained in vitro and in vivo) can be carried out by mass spectrometry analysis. This identification is made possible by virtue of the analysis (determination of the mass) of the peptides generated by enzymatic hydrolysis of the proteins (in general, trypsin). In general, the proteins are isolated according to the methods known to those skilled in the art, prior to the enzymatic digestion. The analysis of the peptides (in hydrolysate form) is carried out by separating of the peptides by HPLC (nano-HPLC) based on their physicochemical properties (reverse phase). The determination of the mass of the peptides thus separated is carried out by ionization of the peptides and either by direct coupling with mass spectrometry (electrospray ESI mode), or after deposition and crystallization in the presence of a matrix known to those skilled in the art (analysis in MALDI mode). The proteins are subsequently identified through the use of appropriate software (for example, Mascot).

According to a third embodiment, step a) described above comprises preparing reaction mixtures, each comprising an ACAA1 or ACAA2 enzyme and a substrate for the enzyme, and step c) described above comprises measuring the enzymatic activity.

The ACAA1 or ACAA2 enzymes can be produced according to customary techniques using Cos-7, CHO, BHK, 3T3 or HEK293 cells. They can also be produced by means of microorganisms such as bacteria (for example, E. coli or B. subtilis), yeasts (for example, Saccharomyces Pichia) or insect cells, such as Sf9 or Sf21.

The enzymes can also be purified from cell homogenates, for example, liver homogenates.

The determination of the enzymatic activity preferably comprises the determination of the acyltransferase activity, by extraction of the fatty acids produced.

Assays for the enzymatic activity of ACAA2 are described in the literature (see, for example, Shindo Y et al., Biochem Pharmacol. 1978; 27(23):2683-8 or Venizelos et al., Pediatr. Res. 1994; 36:111-114).

Thus, the acetyl-coenzyme A acyltransferase 2 activity can, for example, be evaluated in the following way: livers which have not been frozen are homogenized in four volumes of 0.25 M sucrose containing 1 mM of EDTA. Approximately 500 pg of homogenate are incubated in an assay medium of 0.2 ml of potassium chloride at 150 mM, HEPES at 10 mM, pH 7.2, EDTA at 0.1 mM, potassium phosphate buffer at 1 mM, pH 7.2, trismalonate at 5 mM, magnesium chloride at 10 mM, carnitine at 1 mM, bovine serum albumin at 0.15%, ATP at 5 mM and 50 mM of substrate (for example, 3 ketoacyl-CoA), substrate which is radioactive at 5.0×10⁴ cpm. The reaction is carried out for 30 minutes at 25° C. and then stopped by adding 0.2 ml of 0.6 N perchloric acid. The mixture is centrifuged at 2000 g for 10 minutes and the fatty acids which have not reacted in the supernatant are recovered with 2 ml of n-hexane using three extractions. The radioactive degradation products in the aqueous phase are counted. The fatty acid beta-oxidation activity is expressed in nmol/min/liver or any other appropriate unit.

Any other model for assaying the enzymatic activity is possible, in particular using other enzyme substrates, for example fatty acids with longer or shorter chains.

Such methods for assaying enzymatic activity can be used similarly for determining the activity of the ACAA1 enzyme.

The compounds selected by means of the screening methods defined herein can subsequently be tested on other in vitro models and/or in vivo models (in animals or humans) for their effects on acne, seborrhoeic dermatitis or skin disorders associated with hyperseborrhoea.

### Modulators of the Enzyme

A subject of the invention is also the use of a modulator of the human ACAA1 or ACAA2 enzyme, that can be obtained by means of one of the methods above, for the preparation of a medicament for use in the preventive and/or curative treatment of acne, of seborrhoeic dermatitis or of skin disorders associated with hyperseborrhoea.

A method for the preventive and/or curative treatment of acne, of seborrhoeic dermatitis or of skin disorders associated with hyperseborrhoea is thus described herein, said method comprising the administration of a therapeutically effective amount of a modulator of the human ACAA1 or ACAA2 enzyme to a patient requiring such a treatment.

Finally, the invention is directed towards the cosmetic use of a modulator of the human ACAA1 or ACAA2 enzyme, for the aesthetic treatment of greasy skin.

Preferably, the modulator is an inhibitor of the enzyme. The term “inhibitor” refers to a compound or a chemical substance which eliminates or substantially reduces the enzymatic activity of ACAA1 or of ACAA2. The term “substantially” signifies a reduction of at least 25%, preferably of at least 35%, more preferably of at least 50%, and more preferably of at least 70% or 90%. More particularly, it may be a compound which interacts with, and blocks, the catalytic site of the enzyme, such as compounds of the competitive or non-competitive inhibitor type.

A preferred inhibitor interacts with the enzyme in solution at inhibitor concentrations of less than 20 μM, preferably less than 10 μM, more preferably less than 5 μM, less than 1 μM, less than 0.1 μM, more preferably less than 0.01 μM.

The modulator compound may be an anti-ACAA1 or anti-ACAA2 inhibitory antibody, preferably a monoclonal antibody. Advantageously, such an inhibitory antibody is administered in an amount sufficient to obtain a plasma concentration of approximately 0.01 μg per ml to approximately 100 μg/ml, preferably of approximately 1 μg per ml to approximately 5 μg/ml.

The modulator compound may also be a polypeptide, an antisense DNA or RNA polynucleotide, an siRNA or a PNA (peptide nucleic acid, polypeptide chain substituted with purine and pyrimidine bases, the spatial structure of which mimics that of the DNA and enables hybridization thereto).

The modulator compound may also be an aptamer. Aptamers are oligonucleotides which have the ability to recognize virtually all the classes of target molecules with a high affinity and specificity. Such ligands can be isolated by systematic evolution of ligand by exponential enrichment (SELEX) carried out on a library of random sequences, as described by Tuerk and Gold, 1990. The library of random sequences can be obtained by combinatorial chemical synthesis of DNA. In this library, each member is a linear, optionally chemically modified, oligomer of a unique sequence. Possible modifications, uses and advantages of this class of molecules have been reviewed in Jayasena, 1999, Clinical Chemistry 45(9): 1628-1650.

Various ACAA1 or ACAA2 inhibitors can be used. In a nonlimiting manner, mention may be made of 5-(1-hydroxy-2,4,6-heptatriynyl)-2-oxo-1,3-dioxolane-4-heptanoic acid as an inhibitor of acetyl-coenzyme A acyltransferase 2, proposed as a fungicidal treatment (U.S. Pat. No. 4,921,844). The invention comprises the use of such acetyl-coenzyme A acyltransferase 1 or 2-inhibiting compounds for the preventive and/or curative treatment of acne, of seborrhoeic dermatitis or of skin disorders associated with hyperseborrhoea.

Other modulated compounds identified by the screening method described above are also useful.

The modulator compounds are formulated within a pharmaceutical composition, in combination with a pharmaceutically acceptable carrier. These compositions may be administered, for example, orally, enterally, parenterally, or topically. Preferably, the pharmaceutical composition is applied topically. By oral administration, the pharmaceutical composition may be in the form of tablets, gel capsules, sugar-coated tablets, syrups, suspensions, solutions, powders, granules, emulsions, suspensions of microspheres or nanospheres or lipid or polymeric vesicles for controlled release. By parenteral administration, the pharmaceutical composition may be in the form of solutions or suspensions for a drip or for injection.

By topical administration, the pharmaceutical composition is more particularly for use in treating the skin and the mucous membranes and may be in the form of salves, creams, milks, ointments, powders, impregnated pads, solutions, gels, sprays, lotions or suspensions. It may also be in the form of suspensions of microspheres or nanospheres or lipid or polymeric vesicles or polymeric patches or hydrogels for controlled release. This composition for topical application may be in anhydrous form, in aqueous form or in the form of an emulsion. In a preferred variant, the pharmaceutical composition is in the form of a gel, a cream or a lotion.

The composition may comprise an ACAA1- or ACAA2-modulator content ranging from 0.001% to 10% by weight, in particular from 0.01% to 5% by weight, relative to the total weight of the composition.

The pharmaceutical composition may also contain inert additives or combinations of these additives, such as

wetting agents;

flavour enhancers;

preservatives such as para-hydroxybenzoic acid esters;

stabilizers; moisture regulators;

pH regulators;

osmotic pressure modifiers;

emulsifiers;

UV-A and UV-B screens;

and antioxidants, such as alpha-tocopherol, butylhydroxyanisol or butylhydroxytoluene, superoxide dismutase, ubiquinol or certain metal chelating agents.

The following examples illustrate the invention without limiting the scope thereof.

### EXAMPLES

### A. Experimental Data Concerning the ACAA1 Enzyme

### Example 1

### Expression of Acetyl-Coenzyme A Acyltransferase 1 in the Human Sebaceous Gland and in Human Epidermis

Human sebaceous glands were separated from human epidermis by treatment with dispase and dissection under a binocular magnifying lens. Total RNA samples were prepared from the sebaceous glands and from the epidermis.

The expression of the genes was analysed on an Affymetrix station (microfluidic module; hybridization oven; scanner; computer) according to the protocols supplied by the company. Briefly, the total RNA isolated from the tissues is transcribed into cDNA. A biotin-labelled cRNA is synthesized, from the double-stranded cDNA, using T7 polymerase and a precursor NTP conjugated to biotin. The cRNAs are subsequently fragmented into small fragments. All the molecular biology steps are verified using the Agilent “lab on a chip” system in order to confirm that the enzymatic reactions are very efficient. The Affymetrix chip is hybridized with the biotinylated cRNA, rinsed, and subsequently labelled by fluorescence using a Streptavidin-conjugated fluorophore. After washing, the chip is scanned and the results are calculated using the MASS software supplied by Affymetrix. An expression value is obtained for each gene, as is an indication of the significance of the value obtained. The calculation of the significance of the expression is based on the analysis of the signals which are obtained following hybridization of the cRNA of a given gene with a perfect match oligonucleotide versus an oligonucleotide which contains a single mismatch in the central region of the oligonucleotide (see Table 1).

TABLE 1
Measurement of the expression of acetyl-coenzyme A acyltransferase 1 in the epidermis
and in the human sebaceous gland by the use of the Affymetrix chip technology

| | | Expression in | | Significance of | Significance of |
|---|---|---|---|---|---|
| | | the human | Expression in | the expression* | the expression* |
| Affymetrix | | sebaceous | human | in the human | in human |
| identifier | Gene name | gland | epidermis | sebaceous gland | epidermis |
| 202025_x_at | acetyl-Coenzyme A | 200 | 113 | 1 | 1 |
| | acyltransferase 1 | | | | |
| | (peroxisomal 3-oxoacyl- | | | | |
| | Coenzyme A thiolase) | | | | |

*indicator of the significance of the expression of the gene analysed in the sample indicated: presence (=1) or absence (=0).

### Example 2

### Expression of Acetyl-Coenzyme A Acyltransferase 1 in Rat Epidermis After Treatment with a PPARg Agonist

Fuzzy rat epidermal split expression data

The studies are carried out in female Fuzzy rats (Hsd: Fuzzy-fz) ten weeks old at the beginning of the study. The animals are treated at a dose of 1% (PPARg agonist rosiglitazone in solution in acetone) once a day for 8 days. Two hours after the final treatment, the animals are sacrificed by euthanasia and the skin on the back is removed. After incubation in dispase, the epidermis carrying the sebaceous glands is detached from the dermis (epidermal split). After grinding of the samples, the mRNA is prepared using Qiagen columns, in accordance with the supplier's instructions. The material thus prepared is subjected to large-scale transcriptome analysis on an Affymetrix platform. The data are subsequently standardized and, after statistical analysis, the results produced are expressed in arbitrary expression units (see below) accompanied, for each piece of data, by a statistical value for presence of the transcript (presence=1; absence=0).

TABLE 2
Measurement of the expression of ACAA1 in an epidermal split after 8 days of topical
treatment of FUZZY rat females with a PPARγ agonist (rosiglitazone) at 1%

| | | Expression under | | Significance of | Significance of the |
|---|---|---|---|---|---|
| | | the control | Expression after | the expression* | expression* after |
| Affymetrix | | condition | treatment with | under the | treatment with |
| identifier | Gene name | (DMSO) | 1% rosiglitazone | control condition | 1% rosiglitazone |
| 1387783_a_at | acetyl-Coenzyme A | 160 | 455 | 1 | 1 |
| | acyltransferase 1 | | | | |

*indicator of the significance of the expression of the gene analysed in the sample indicated: presence (=1) or absence (=0).

### B. Experimental Data Concerning the ACAA2 Enzyme

### Example 3

### Expression of Acetyl-Coenzyme A Acyltransferase 2 in the Human Sebaceous Gland and in Human Epidermis

Human sebaceous glands were separated from human epidermis by treatment with dispase and dissection under a binocular magnifying lens. Total RNA samples were prepared from the sebaceous glands and from the epidermis.

The expression of the genes was analysed on an Affymetrix station (microfluidic module; hybridization oven; scanner; computer) according to the protocols supplied by the company. Briefly, the total RNA isolated from the tissues is transcribed into cDNA. A biotin-labelled cRNA is synthesized, from the double-stranded cDNA, using T7 polymerase and a precursor NTP conjugated to biotin. The cRNAs are subsequently fragmented into small fragments. All the molecular biology steps are verified using the Agilent “lab on a chip” system in order to confirm that the enzymatic reactions are very efficient. The Affymetrix chip is hybridized with the biotinylated cRNA, rinsed, and subsequently labelled by fluorescence using a Streptavidin-conjugated fluorophore. After washing, the chip is scanned and the results are calculated using the MASS software supplied by Affymetrix. An expression value is obtained for each gene, as is an indication of the significance of the value obtained. The calculation of the significance of the expression is based on the analysis of the signals which are obtained following hybridization of the cRNA of a given gene with a perfect match oligonucleotide versus an oligonucleotide which contains a single mismatch in the central region of the oligonucleotide (see Table 3).

TABLE 3
Measurement of the expression of acetyl-coenzyme A acyltransferase 2 in the epidermis
and in the human sebaceous gland via the use of the Affymetrix chip technology

| | | Expression in | | Significance of the | Significance of |
|---|---|---|---|---|---|
| | | the human | Expression | expression* in the | the expression* |
| Affymetrix | | sebaceous | in human | human sebaceous | in human |
| identifier | Gene name | gland | epidermis | gland | epidermis |
| 202003_s_at | acetyl-Coenzyme A | 363 | 70 | 1 | 0 |
| | acyltransferase 2 | | | | |

*indicator of the significance of the expression of the gene analysed in the sample indicated: presence (=1) or absence (=0).

### Example 4

### Expression of Acetyl-Coenzyme A Acyltransferase 2 in the Human Sebaceous Gland and in Human Epidermis

The samples of epidermis and of human sebaceous gland were prepared by laser microdissection from three lifts of healthy human skin (female donors).

The expression of the messenger RNA encoding the ACAA2 protein was analysed by quantitative RT-PCR (qRT-PCR) using the microfluidics cards technology developed by Applied Biosystems.

The Ct corresponds to the number of PCR cycles which makes it possible to choose the same level of fluorescence for all the samples. The level of expression is represented in each tissue by the mean of the Cts and the standard deviation obtained on the three donors.

The differential expression between the two tissues is measured via a mean induction factor (I.F) for the sebaceous gland with respect to the epidermis after standardization of the Cts via the expression of the three housekeeping genes (ribosomal 18S RNA, glyceraldehyde 3-phosphate dehydrogenase GAPDH, beta-actin).

TABLE 4
qRT-PCR measurement of the expression of acetyl-coenzyme A acyltransferase 2 in the epidermis and
the human sebaceous gland via the use of the microfluidic cards technology (Applied Biosystems)

| | Number of cycles necessary | | Number of cycles necessary | | Mean induction factor |
|---|---|---|---|---|---|
| | for detecting the mean | | for detecting the mean | | for expression in the |
| | expression in the human | Standard | expression in human | Standard | sebaceous gland versus |
| Gene name | sebaceous gland (Ct) | deviation | epidermis (Ct) | deviation | human epidermis (I.F) |
| acetyl-Coenzyme A | 25 | 0.76 | 30 | 2.17 | 35 |
| acyltransferase 2 | | | | | |

### Example 5

### Expression of Acetyl-Coenzyme A Acyltransferase 2 in Human Sebocytes in Primary Culture

a. Isolation and Culture of Human Sebocytes

Human sebocytes are cultured using lifts from healthy human donors according to the method described by Xia et al. (J Invest Dermatol. 1989 September; 93(3):315-21) after separation of the epidermis from the dermis through the action of dispase and microdissection of the sebaceous glands under binocular magnifying lenses.

The sebaceous glands are seeded in 6-well plates on a feeder layer of mitomycin-treated 3T3 fibroblasts in DMEM-Ham's F12 (3:1) medium supplemented with 10% foetal calf serum (FCS); 10 ng/ml of epidermal growth factor (EGF); 10¹⁰ M cholera toxin (CT); 0.5 μg/ml of hydrocortisone (HC); 5 μg/ml of insulin (INS); 2 mM L-glutamine (Gln); 100 IU/ml of penicillin-streptomycin (PS).

The first foci of human sebocytes appear 3 days after seeding of the glands.

The cells are then treated for 6 days with the sebogenic cocktail corresponding to the combination of PPARγ agonist rosiglitazone (1 μM) and the androgen R1881 (10 nM), or with dimethyl sulphoxide (DMSO) used as carrier.

b. PCR Expression Data

The expression of the messenger RNA encoding the ACAA2 protein was analysed by qRT-PCR using the microfluidics cards technology developed by Applied Biosystems, as described above (Example 2), on a culture of human sebocytes corresponding to one donor.

The level of expression (Ct) is represented for each treatment condition.

The induction of ACAA2 expression by the sebogenic cocktail is measured via an induction factor (I.F) versus the DMSO control after standardization of the Cts via the expression of the three housekeeping genes (ribosomal 18S RNA, glyceraldehyde 3-phosphate dehydrogenase GAPDH, beta-actin).

TABLE 5
qRT-PCR measurement of the expression of ACAA2 in a primary culture of human sebocytes treated for
6 days with the sebogenic cocktail (combination of 1 μM PPARγ agonist rosiglitazone; 10
nM androgen R1881) or with DMSO, via the use of the microfluidic cards technology (Applied Biosystems)

| | | Level of expression | Induction factor for |
|---|---|---|---|
| | Level of expression | in human sebocytes | expression under the condition |
| | in human sebocytes | treated with the sebo- | treated with the sebogenic cocktail |
| Gene name | treated with DMSO (Ct) | genic cocktail (Ct) | versus the DMSO condition (I.F) |
| acetyl-Coenzyme A | 30 | 27 | 7.4 |
| acyltransferase 2 | | | |

### Example 6

### Expression of Acetyl-Coenzyme A Acyltransferase 2 in the Rat Preputial Gland

Primary cultures of rat preputial gland sebocytes (Rosenfield et al., J. Invest. Dermatol. 1999; 112:226-32) were used to evaluate differentiation cocktails such as the combination of PPARγ and an androgen receptor agonist. After seeding on 24-well plates, the preputial cells are cultured for 3 days in DMEM medium containing 10% of foetal calf serum (FCS), 10⁻¹⁰ M of cholera toxin (CT), 10¹⁰ M of cortisol, 5 μg/ml of insulin and antibiotics. The cells are then cultured in a serum-free medium (Cellgro complete medium) and treated with the PPARγ agonist (rosiglitazone, 100 nM) and the androgen receptor agonist (R1181, 1 nM) for 3 to 9 days with the medium being changed every 2 days. The cells are recovered on the 9^{th} day and the large-scale analysis of the gene expression is carried out by means of Affymetrix RAE230A chips.

TABLE 6
Measurement of the expression of acetyl-Coenzyme A acyltransferase 2 in preputial gland
cells in culture in response to a cocktail of an androgen (R1881 at 1 nM) and of a PPARγ
ligand (rosiglitazone at 100 nM) via the use of the Affymetrix chip technology. The mixture
is known to induce cell differentiation characterized by increased lipogenesis

| | | Expression under | Expression after | Significance of | Significance of the |
|---|---|---|---|---|---|
| | | the control | treatment with | the expression* | expression* after |
| Affymetrix | | condition | R1881 and | under the | treatment with R1881 |
| identifier | Gene name | (DMSO) | rosiglitazone | control condition | and rosiglitazone |
| 1386880_at | acetyl-Coenzyme A | 293 | 402 | 1 | 1 |
| | acyltransferase 2 | | | | |

*Indicator of the significance of the expression of the gene analysed in the sample indicated: presence (=1) or absence (=0).

1.-30. (canceled)

31. An in vitro or in vivo method for screening for candidate compounds for the preventive and/or curative treatment of acne, of seborrhoeic dermatitis or of skin disorders associated with hyperseborrhoea, comprising determining the ability of a compound to modulate the expression or the activity of acetyl-coenzyme A acyltransferase 1 (ACAA1) or of acetyl-coenzyme A acyltransferase 2 (ACAA2) or the expression of the gene thereof, or the activity of at least one of the promoters thereof.

32. An in vitro method for screening for candidate compounds for the preventive and/or curative treatment of acne, of seborrhoeic dermatitis or of skin disorders associated with hyperseborrhoea as defined by claim 31, comprising the following steps:
a. preparing at least two biological samples or reaction mixtures;
b. contacting one of the samples or reaction mixtures with one or more of the test compounds;
c. measuring the expression or the activity of the acetyl-coenzyme A acyltransferase 1 or acetyl-coenzyme A acyltransferase 2 proteins, the expression of the gene thereof or the activity of at least one of the promoters thereof, in the biological samples or reaction mixtures; and
d. selecting the compounds for which a modulation of the expression or of the activity of the acetyl-coenzyme A acyltransferase 1 or acetyl-coenzyme A acyltransferase 2 proteins, or a modulation of the expression of the gene thereof or a modulation of the activity of at least one of the promoters thereof, is measured in the sample or the mixture treated in b) compared with the untreated sample or with the untreated mixture.

33. The in vitro method as defined by claim 32, wherein the compounds selected in step d) inhibit the expression or the activity of the acetyl-coenzyme A acyltransferase 1 or acetyl-coenzyme A acyltransferase 2 proteins, the expression of the gene thereof or the activity of at least one of the promoters thereof.

34. The in vitro method as defined by claim 32, wherein the biological samples are cells transfected with a reporter gene functionally linked to all or part of the promoter of the gene encoding acetyl-coenzyme A acyltransferase 1 or acetyl-coenzyme A acyltransferase 2, and step c) comprises measuring the expression of said reporter gene.

35. The in vitro method as defined by claim 32, wherein the biological samples are cells expressing the gene encoding acetyl-coenzyme A acyltransferase 1 or acetyl-coenzyme A acyltransferase 2, and step c) comprises measuring the expression of said gene.

36. The in vitro method as defined by claim 34, wherein the cells comprise sebocytes.

37. The in vitro method as defined by claim 35, wherein the cells comprise cells transformed with a heterologous nucleic acid encoding acetyl-coenzyme A acyltransferase 1 or acetyl-coenzyme A acyltransferase 2.

38. The in vitro method as defined by claim 32, wherein the expression of the gene is determined by measuring the level of transcription of said gene.

39. The in vitro method as defined by claim 32, wherein the expression of the gene is determined by measuring the level of translation of said gene.

40. The in vitro method as defined by claim 32, wherein step a) comprises preparing reaction mixtures, each comprising an acetyl-coenzyme A acyltransferase 1 or acetyl-coenzyme A acyltransferase 2 enzyme and a substrate for the enzyme, and step c) comprises measuring the enzymatic activity.

41. The in vitro method as defined by claim 40, wherein the determination of the enzymatic activity comprises the determination of the acyltransferase activity, by extraction of the fatty acids produced.

42. A medicament useful for the preventive and/or curative treatment of acne, of seborrhoeic dermatitis or of skin disorders associated with hyperseborrhoea, comprising a modulator of the human acetyl-coenzyme A acyltransferase 1 or acetyl-coenzyme A acyltransferase 2 enzyme obtained by means of the method as defined by claim 32.

43. The medicament as defined by claim 42, wherein the modulator comprises an inhibitor of the enzyme.

44. The medicament as defined by claim 43, wherein the modulator comprises a compound which interacts with, and blocks, the catalytic site of the enzyme.

45. A regime or regimen for the aesthetic treatment of greasy skin, comprising administering to an individual in need of such treatment, a thus effective amount of a modulator of the human acetyl-coenzyme A acyltransferase 1 or acetyl-coenzyme A acyltransferase 2 enzyme.

46. An in vitro method for diagnosing or monitoring the development of acne, seborrhoeic dermatitis or a skin disorder associated with hyperseborrhoea in an individual, comprising comparing the expression or the activity of the acetyl-coenzyme A acyltransferase 1 or acetyl-coenzyme A acyltransferase 2 proteins, the expression of the gene thereof or the activity of at least one promoter thereof, in a biological sample from an individual, with respect to a biological sample from a control individual.

47. The in vitro method as defined by claim 46, wherein the expression of the proteins is determined by assaying this protein by immunoassay.

48. The in vitro method as defined by claim 47, wherein the immunoassay comprises an ELISA assay.

49. The in vitro method as defined by claim 46, wherein the expression of the genes is determined by measuring the amount of corresponding mRNA.

50. An in vitro method for determining an individual's susceptibility to developing acne, seborrhoeic dermatitis or a skin disorder associated with hyperseborrhoea, comprising comparing the expression or the activity of the acetyl-coenzyme A acyltransferase 1 or acetyl-coenzyme A acyltransferase 2 proteins, the expression of the gene thereof or the activity of at least one of the promoters thereof, in a biological sample from an individual, with respect to a biological sample from a control individual.

51. The in vitro method as defined by claim 50, wherein the expression of the proteins is determined by assaying this protein by means of an immunoassay.

52. The in vitro method as defined by claim 51, wherein the immunoassay comprises an ELISA assay or a radioimmunoassay.

53. The in vitro method as defined by claim 50, wherein the expression of the genes is determined by measuring the amount of corresponding mRNA.

54. A marker for screening for candidate PPAR modulators for the treatment of acne, of seborrhoeic dermatitis or a skin disorder associated with hyperseborrhoea, comprising an acetyl-coenzyme A acyltransferase 1 or acetyl-coenzyme A acyltransferase 2 gene or an acetyl-coenzyme A acyltransferase 1 or acetyl-coenzyme A acyltransferase 2 protein.

55. The marker as defined by claim 54, for determining the ability of a PPAR modulator to modulate the expression or the activity of acetyl-coenzyme A acyltransferase 1 or acetyl-coenzyme A acyltransferase 2 or the expression of the gene thereof or the activity of at least one of the promoters thereof.

56. The marker as defined by claim 54, wherein the PPAR modulator comprises a PPARγ modulator.

57. The marker as defined by claim 54, wherein the modulator comprises a PPAR receptor agonist.

58. A marker for screening for candidate AR (androgen receptor) modulators for the treatment of acne, of seborrhoeic dermatitis or of a skin disorder associated with hyperseborrhoea, comprising an acetyl-coenzyme A acyltransferase 1 or acetyl-coenzyme A acyltransferase 2 gene or an acetyl-coenzyme A acyltransferase 1 or acetyl-coenzyme A acyltransferase 2 protein.

59. The marker as defined by claim 58, for determining the ability of an AR modulator to modulate the expression or the activity of acetyl-coenzyme A acyltransferase 1 or acetyl-coenzyme A acyltransferase 2 or the expression of the gene thereof or the activity of at least one of the promoters thereof.

60. The marker as defined by claim 58, wherein the modulator comprises an androgen receptor agonist.
